(19) United States
(12) Patent Application Publication
(10) Pub. No.: US 2011/0245173 A1
(43) Pub. Date: Oct. 6, 2011

(54) Long-Acting GLP-1 Derivatives, and Methods of Treating Cardiac Dysfunction

(76) Inventors: William W. Bachovchin, Cambridge, MA (US); Hung-sen Lai, Andover, MA (US); David George Sanford, Reading, MA (US)

(21) Appl. No.: 12/995,813

(22) PCT Filed: Jun. 3, 2009
(86) PCT No.: PCT/US2009/046070

§ 371 Date: Jun. 7, 2011

Related U.S. Application Data
(60) Provisional application No. 61/058,423, filed on Jun. 3, 2008.

(51) Int. Cl. A61K 38/22; C07K 14/575; A61P 9/00

(52) U.S. Cl. 514/11.7; 530/324

(57) ABSTRACT

The present invention generally provides polypeptide analogues of GLP-‘(9-34) and GLP-1 (9-36) that have increased in vivo half-lives resulting from reduced susceptibility to proteolytic enzymes. Other aspects of the invention relate to methods of using the polypeptide analogues described herein for treating cardiac dysfunction and other heart-related maladies. Yet another aspect of the present invention relates lo formulations comprising the polypeptide analogues described herein.

### RELATED APPLICATIONS

This application claims the benefit of priority to U.S. Provisional Patent Application Ser. No. 61/058,423, filed Jun. 3, 2008.

### BACKGROUND OF THE INVENTION

Polypeptide and peptide therapeutics, such as hormones, cytokines and growth factors, are widely used in medical practice. Their ease of production, either by recombinant DNA technology or peptide synthesizers, ensures their continued use in a variety of circumstances in the years to come. Certain native polypeptides, however, can be inactivated rapidly in vivo via proteolysis or isomerization. Such inactivation can be inconvenient in cases where it is desired to maintain a consistent or sustained blood level of the therapeutic over a period of time, as repeated administrations are then necessary. In certain instances, one or more of the proteolytic products of the polypeptide can be antagonistic to the activity of the intact polypeptide. In these cases, administration of additional therapeutic agent alone may not be sufficient to overcome the antagonist effect of the proteolytic products.

Glucagon-like peptide 1 (GLP-1) is an endogenous physiological insulinotropic and glucagonostatic 30-amino-acid peptide incretin hormone that acts in a self-limiting mechanism and is responsible for approximately 80% of the incretin effect (Gutniak et al. (1992) N. Engl. J. Bled. 326:1316-1322). This multifunctional hormone is released from the L-cells in the intestine (primarily in the ileum and colon) and serves to augment the insulin response after an oral intake of glucose or fat (Mosjov, S., In J. Peptide Protein Research, 40:333-343 (1992); Gutniak et. al, supra; Mosjov et al. (1988) J. Clin Invest 79:616; Schmidt et al. (1985) Diabetologia 28:704; and Kreymann et al. (1987) Lancet 2:1300). GLP-1 lowers glucagon concentrations, stimulates (pro)insulin biosynthesis, enhances insulin sensitivity, stimulates the insulin-independent glycogen synthesis, retards gastric emptying, reduces appetite, and leads to liver glucagon breakdown suppression, up-regulation of islet cell proliferation, and neogenesis. Infusion of GLP-1 has been shown to normalize the level of HbA1C and enhance the ability of β-cells to sense and respond to increased glucose levels in humans with impaired glucose tolerance.

Dipeptidyl peptidase IV (DPP-IV) is an enzyme naturally present in the body that works rapidly in the serum to cleave the native GLP-1 (7-36) N-terminal dipeptide [His⁷-Ala⁸], effectively curtailing the biological activity of GLP-1. The cleavage product of DPP IV-mediated degradation is GLP-1 (9-36), a compound at one time believed to have little or no biological activity. Recently, the DPP-IV cleavage product GLP-1 (9-36) has been reported to have some (i.e., about 20% of the activity of the native molecule) glucose-lowering effects in peripheral tissues. Deacon, C. F., et al. Am J Physiol Endocrinol Metab. 282(4):E873-E879 (2002). This effect is not dependent upon insulin release and the receptor(s) mediating this effect have not been identified.

Remarkably, GLP-1 (9-36) has also been shown to be as potent as the native molecule in reversing cardiac dysfunction in the pacing-induced canine heart-failure model, an effect that is at least partially dependent upon enhanced myocardial glucose uptake. Nikolaides, L. A., et al. Circulation (Supplement III) 110: III680 (2004). Again this effect is independent of the GLP-1 receptor. Recently, Elahi et al. reported that GLP-1 (9-36) lowers fasting blood glucose in diabetic animals. Elahi, D. et al., Obesity 16(7): 1501-1509 (2008). Whether or not this effect is related to the cardio protective, or the glucose lowering effects described above is not clear. Nevertheless, it is appears that GLP-1 (9-36) has biological activities independent of the GLP-1 (7-36) receptor and that these activities are therapeutically useful. However, like GLP-1 (7-36), GLP-1 (9-36) has a very short half-life in vivo (T₁₁₂ is about 2-4 minutes) which may limit its usefulness as a therapeutic. Therefore, developing analogues of GLP-1 (9-36) that significantly extend its lifetime in vivo would be useful in treating cardiac dysfunction.

### SUMMARY OF THE INVENTION

The present invention generally provides polypeptide analogues of GLP-1 (9-34) and GLP-1 (9-36) that have longer in vivo half-lives than the native polypeptides.

In one aspect, the present invention relates to a polypeptide comprising:

a base amino acid sequence at least 90% identical to GLP-1 (9-34) or GLP-1 (9-36) (SEQ ID NOS: 1 and 2), wherein the analogue has a longer in vivo half-life than GLP-1 (9-34) or GLP-1 (9-36).

In another aspect, the present invention relates to polypeptide analogue comprising:
a) a base amino acid sequence at least 90% identical to GLP-1 (9-34) or GLP-1 (9-36) (SEQ ID NOS: 1 and 2); and
b) one to fifteen amino acid residues attached to the carboxy terminus of the base amino acid sequence, wherein the analogue has a longer in vivo half-life than GLP-1 (9-34) or GLP-1 (9-36).

In a further aspect, the present invention relates to retro-inverso polypeptide analogue comprising: a base amino acid sequence at least 90% identical to GLP-1 (9-34) or GLP-1 (9-36) (SEQ ID NOS: 1 and 2) comprising D-amino acids assembled in reversed order along the peptide chain, wherein the analogue has a longer in vivo half-life than GLP-1 (9-34) or GLP-1 (9-36).

In another aspect, the present invention relates to a retro-inverso polypeptide analogue comprising:
a) a base amino acid sequence at least 90% identical to GLP-1 (9-34) or GLP-1 (9-36) (SEQ ID NOS: 1 and 2) comprising D-amino acids assembled in reversed order along the peptide chain; and
b) one to fifteen amino acid residues attached to the amino terminus of the base amino acid sequence, wherein the analogue has a longer in vivo half-life than GLP-1 (9-34) or GLP-1 (9-36).

In another aspect, the present invention relates to a polypeptide analogue comprising:

a base amino acid sequence at least 90% identical to GLP-1 (9-34) or GLP-1 (9-36) (SEQ ID NOS: 1 and 2); wherein the amino acid residue corresponding to position 9 of GLP-1 is an amino acid analogue having a tetrasubstituted C_{β} carbon; and the analogue has longer in vivo half-life than GLP-1 (9-34) or GLP-1 (9-36).

In yet another aspect, the present invention relates to a polypeptide analogue comprising:
a) a base amino acid sequence at least 90% identical to one of GLP-1 (9-34), GLP-1 (9-36), (SEQ ID NOS: 1 and 2); wherein the amino acid residue corresponding to position 9 of GLP-1 is an amino acid analogue having a tetrasubstituted C_{β} carbon; and
b) one to fifteen amino acid residues attached to the carboxy terminus of the base amino acid sequence, wherein the analogue has a longer in vivo half-life than GLP-1 (9-34) or GLP-1 (9-36).

Another aspect of the present invention provides formulations comprising any of the polypeptide analogues of the invention and pharmaceutically acceptable excipients.

Other aspects of the invention are to methods for treating the cardiac disorders (e.g., cardiac dysfunction or ischemia-reperfusion injury) disclosed herein by administering a therapeutically effective amount of one or more of any of the polypeptide analogues disclosed. The polypeptide analogues can be administered alone, or can be administered as part of a therapeutic regimen including other therapies appropriate to the specific cardiac dysfunction.

### BRIEF DESCRIPTION OF THE FIGURES

FIG. 1 depicts exemplary modifications that may be made to an amino acid sequence in accordance with the present invention. The variables R¹, R², R³, and R⁴ may represent amino acid side chains, and Xaa may represent any amino acid residue.

FIG. 2 depicts exemplary GLP-1 (9-36) analogues with C-terminal extensions.

FIG. 3 shows the plasma lifetime of exemplary GLP-1 (9-36) analogues with C-terminal extensions.

### DETAILED DESCRIPTION OF THE INVENTION

### Long Lived GLP-1 (9-34) and GLP-1 (9-36) Analogues

An aspect of the present invention relates to polypeptide analogues of GLP-1 (9-34) and GLP-1 (9-36) that have increased in vivo half-lives, e.g., resulting from reduced susceptibility to cleavage by proteolytic enzymes. The polypeptide analogues of the invention can be rendered resistant to cleavage by proteinases selected from: an aminopeptidase (EC 3.4.11.-), a dipeptidase (EC 3.4.13.-), a dipeptidyl-peptidase or tripeptidyl peptidase (EC 3.4.14.-), a peptidyl-dipeptidase (EC 3.4.15.-), a serine-type carboxypeptidase (EC 3.4.16.-), a metallocarboxypeptidase (EC 3.4.17.-), a cysteine-type carboxypeptidase (EC 3.4.18.-), an omegapeptidase (EC 3.4.19.-), a serine proteinase (EC 3.4.21.-), a cysteine proteinase (EC 3.4.22.-), an aspartic proteinase (EC 3.4.23.-), a metallo proteinase (EC 3.4.24.-), or a proteinase of unknown mechanism (EC 3.4.99.-). The EC designation following each class of proteinase is that used in the recommendation of the International Union of Biochemistry and Molecular Biology (1984), and these subclass headings are provided here for reference.

To further illustrate the exemplary proteinases for which the polypeptide analogues of the invention are contemplated, an non-exhaustive list of proteinases include: leucyl aminopeptidase, membrane alanine aminopeptidase, cystinyl aminopeptidase, tripeptide aminopeptidase, prolyl aminopeptidase, aminopeptidase B, glutamyl aminopeptidase, Xaa-Pro aminopeptidase, bacterial leucyl aminopeptidase, clostridial aminopeptidase, cytosol alanyl aminopeptidase, lysyl aminopeptidase, Xaa-Trp aminopeptidase, tryptophanyl aminopeptidase, methionyl aminopeptidase, D-stereospecific aminopeptidase, aminopeptidase Ey, vacuolar aminopeptidase I, Xaa-His dipeptidase, Xaa-Arg dipeptidase, Xaa-methyl-His dipeptidase, Cys-Gly dipeptidase, Glu-Glu dipeptidase, Pro-Xaa dipeptidase, Xaa-Pro dipeptidase, Met-Xaa dipeptidase, non-stereospecific dipeptidase, cytosol non-specific dipeptidase, membrane dipeptidase, β-Ala-His dipeptidase, Dipeptidyl-peptidase I (DPP I), Dipeptidyl-peptidase II (DPP II), Dipeptidyl-peptidase III (DPP III), Dipeptidyl-peptidase IV(DPP IV), Dipeptidyl-dipeptidase, Tripeptidyl-peptidase I, Tripeptidyl-peptidase II, Xaa-Pro dipeptidyl-peptidase, peptidyl-dipeptidase A, peptidyl-dipeptidase B, peptidyl-dipeptidase Dcp, lysosomal Pro-X carboxypeptidase, Serine-type D-Ala-D-Ala carboxypeptidase, carboxypeptidase C, carboxypeptidase D, carboxypeptidase A, carboxypeptidase B, lysine(arginine) carboxypeptidase, Gly-X carboxypeptidase, alanine carboxypeptidase, muramoylpentapeptide carboxypeptidase, carboxypeptidase H, glutamate carboxypeptidase, carboxypeptidase M, muramoyltetrapeptide carboxypeptidase, zinc D-Ala-D-Ala carboxypeptidase, carboxypeptidase A2, membrane Pro-X carboxypeptidase, tubulinyl-Tyr carboxypeptidase, carboxypeptidase T, thermostable carboxypeptidase 1, carboxypeptidase U, glutamate carboxypeptidase II, metallocarboxypeptidase D, cysteine-type carboxypeptidase, acylaminoacyl-peptidase, peptidyl-glycinamidase, pyroglutamyl-peptidase I, beta-aspartyl-peptidase, pyroglutamyl-peptidase II, N-formylmethionyl-peptidase, pteroylpoly-gamma-glutamate carboxypeptidase, gamma-glutamyl hydrolase, gamma-D-glutamyl-meso-diamino-pimelate peptidase I, chymotrypsin, chymotrypsin C, metridin, trypsin, thrombin, coagulation factor Xa, plasmin, enteropeptidase, acrosin, alpha-lytic endopeptidase, glutamyl endopeptidase, cathepsin G, coagulation factor VIIa, coagulation factor Ixa, cucumisin, prolyl oligopeptidase, coagulation factor XIa, brachyurin, plasma kallikrein, tissue kallikrein, pancreatic elastase, leukocyte elastase, coagulation factor XIIa, chymase, complement component C1r, complement component C1s, classical-complement pathway C3/C5 convertase, complement factor I, complement factor D, alternative-complement pathway C3/C5 convertase, cerevisin, hypodermin C, lysyl endopeptidase, endopeptidase La, gamma-renin, venombin AB, leucyl endopeptidase, tryptase, scutelarin, kexin, subtilisin, oryzin, proteinase K, thermomycolin, thermitase, endopeptidase So, T-plasminogen activator, protein C (activated), pancreatic endopeptidase E, pancreatic elastase II, IgA-specific serine endopeptidase, U-plasminogen activator, venombin A, furin, myeloblastin, semenogelase, granzyme A, granzyme B, streptogrisin A, streptogrisin B, glutamyl endopeptidase II, oligopeptidase B, limulus clotting factor C, limulus clotting factor B, limulus clotting enzyme, omptin, repressor lexA, signal peptidase I, togavirin, flavirin, endopeptidase Clp, proprotein convertase 1, proprotein convertase 2, snake venom factor V activator, lactocepin, cathepsin B, papain, ficain, chymopapain, asclepain, clostripain, streptopain, actimidain, cathepsin L, cathepsin H, calpain, cathepsin T, glycyl endopeptidase, cancer procoagulant, cathepsin S, picomain 3C, picornain 2A, caricain, ananain, stem bromelain, fruit bromelain, legumain, histolysain, caspase-1, gingipain R, cathepsin K, pepsin A, pepsin B, gastricsin, chymosin, cathepsin D, neopenthesin, renin, retropepsin, pro-opiomelanocortin converting enzyme, aspergillopepsin I, aspergillopepsin II, penicillopepsin, rhizopuspepsin, endothiapepsin, mucoropepsin, candidapepsin, saccharopepsin, rhodotorulapepsin, physaropepsin, acrocylindropepsin, polyporopepsin, pycnoporopepsin, scytalidopepsin A, scytalidopepsin B, xanthomonapepsin, cathepsin E, barrierpepsin, signal peptidase II, pseudomonapepsin, plasmepsin I, plasmepsin II, phytepsin, atrolysin A, microbial collagenase, leucolysin, interstitial collagenase, neprilysin, envelysin, IgA-specific metalloendopeptidase, procollagen N-endopeptidase, thimet oligopeptidase, neurolysin, stromelysin 1, meprin A, procollagen C-endopeptidase, peptidyl-Lys metalloendopeptidase, astacin, stromelysin 2, matrilysin, gelatinase A, aeromonolysin, pseudolysin, thermolysin, bacillolysin, aureolysin, coccolysin, mycolysin, beta-lytic metalloendopeptidase, peptidyl-Asp metalloendopeptidase, neutrophil collagenase, gelatinase B, leishmanolysin, saccharolysin, autolysin, deuterolysin, serralysin, atrolysin B, atrolysin C, atroxase, atrolysin E, atrolysin F, adamalysin, horrilysin, ruberlysin, bothropasin, bothrolysin, ophiolysin, trimerelysin I, trimerelysin II, mucrolysin, pitrilysin, insulysin, O-sialoglycoprotein endopeptidase, russellysin, mitochondrial intermediate peptidase, dactylysin, nardilysin, magnolysin, meprin B, mitochondrial processing peptidase, macrophage elastase, choriolysin L, choriolysin H, tentoxilysin, bontoxilysin, oligopeptidase A, endothelin-converting enzyme 1, fibrolase, jararhagin, fragilysin, and multicatalytic endopeptidase complex.

In certain embodiments, the present invention relates to a polypeptide comprising: a base amino acid sequence at least 90% identical to GLP-1 (9-34) or GLP-1 (9-36) (SEQ ID NOS: 1 and 2), wherein the analogue has a longer in vivo half-life than GLP-1 (9-34) or GLP-1 (9-36).

Another aspect of the present invention relates to C-terminal modifications of GLP-1 (9-34) or GLP-1 (9-36) to prolong the biological half-life of the polypeptides in vivo. In certain embodiments, the present invention relates to a polypeptide analogue comprising:
a) a base amino acid sequence at least 90% identical to GLP-1 (9-34) or GLP-1 (9-36) (SEQ ID NOS: 1 and 2); and
b) one to fifteen amino acid residues attached to the carboxy terminus of the base amino acid sequence, wherein the analogue has a longer in vivo half-life than GLP-1 (9-34) or GLP-1 (9-36).

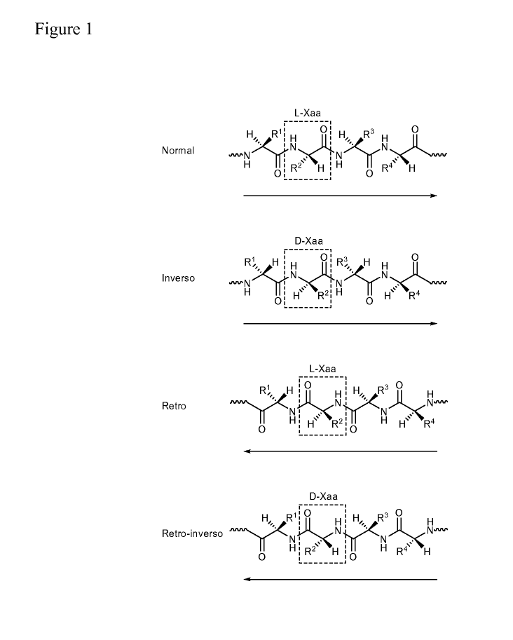

Another aspect of the present invention relates to retro-inverso polypeptide analogues of GLP-1 (9-34) and GLP-1 (9-36) whereby the use of complementary D-amino acid enantiomers constitutes an inversion of the chirality of the amino acid residues in the native sequence (inversion modification), and whereby said D-amino acids are attached in a peptide chain such that the sequence of residues in the resulting analogue is exactly opposite of that in the native GLP-1 analogue (retro modification). (See FIG. 1)

In certain embodiments, the present invention relates to a retro-inverso polypeptide analogue comprising: a base amino acid sequence at least 90% identical to GLP-1 (9-34) or GLP-1 (9-36) (SEQ ID NOS: 1 and 2) comprising D-amino acids assembled in reversed order along the peptide chain, wherein the analogue has a longer in vivo half-life than GLP-1 (9-34) or GLP-1 (9-36).

In certain embodiments, the present invention relates to a retro-inverso polypeptide analogue comprising:
a) a base amino acid sequence at least 90% identical to GLP-1 (9-34) or GLP-1 (9-36) (SEQ ID NOS: 1 and 2) comprising D-amino acids assembled in reversed order along the peptide chain; and
b) one to fifteen amino acid residues attached to the amino terminus of the base amino acid sequence, wherein the analogue has a longer in vivo half-life than GLP-1 (9-34) or GLP-1 (9-36).

In certain embodiments, the retro-inverso polypeptide analogue comprises D-allo amino acids. In certain embodiments, the present invention makes use of complementary diastereometric D-allo amino acids as a conservative substitution for threonine and isoleucine residues within the GLP-1 analogues disclosed herein.

In certain embodiments, the retro-inverso polypeptide analogue has only allo amino acids at positions corresponding to D-threonine and D-isoleucine.

Another aspect of the present invention relates to a polypeptide analogue comprising:

a base amino acid sequence at least 90% identical to GLP-1 (9-34) or GLP-1 (9-36) (SEQ ID NOS: 1 and 2); wherein the amino acid residue corresponding to position 9 of GLP-1 is an amino acid analogue having a tetrasubstituted C_{β} carbon; and the analogue has longer in vivo half-life than GLP-1 (9-34) or GLP-1 (9-36).

In certain embodiments, the present invention relates to a polypeptide analogue comprising:
a) a base amino acid sequence at least 90% identical to one of GLP-1 (9-34), GLP-1 (9-36), (SEQ ID NOS: 1 and 2); wherein the amino acid residue corresponding to position 9 of GLP-1 is an amino acid analogue having a tetrasubstituted C_{β} carbon; and
b) one to fifteen amino acid residues attached to the carboxy terminus of the base amino acid sequence, wherein the analogue has a longer in vivo half-life than GLP-1 (9-34) or GLP-1 (9-36).

In certain embodiments, the amino acid residue corresponding to position 9 of GLP-1 is represented by the following formula:

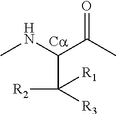

wherein:
R₁ and R₂ each independently represent a lower alkyl, heteroalkyl, cycloalkyl, heterocycloalkyl, aryl, alkoxyl, carbonyl, carboxamide, halogen, hydroxyl, amine, or cyano, or R₁ and R₂ taken together form a ring of 4-7 atoms;
R₃ represents a lower alkyl, a heteroalkyl, amino, alkoxyl, halogen, carboxamide, carbonyl, cyano, thiol, thioalkyl, acylamino, nitro, azido, sulfate, sulfonate, sulfonamido, —(CH₂)ₘ—R₄, —(CH₂)ₘ—OH, —(CH₂)ₘ—COOH, —(CH₂)ₘ—O-lower alkyl, —(CH₂)ₘ—O-lower alkenyl, —(CH₂)ₙ—O—(CH₂)ₘ—R₄, (CH₂)ₘ—S-lower alkyl, —(CH₂)ₘ—S-lower alkenyl, —(CH₂)ₙ—S—(CH₂)ₘ—R₄, —(CH₂)ₘ—N—C(═NH)NH₂, —(CH₂)ₘ—C(═O)NH₂, or —(CH₂)ₘ—NH₂;
R₄ represents, independently for each occurrence, an aryl, aralkyl, cycloalkyl, cycloalkenyl, or non-aromatic heterocyclyl; and m is 0, 1 or 2.

In certain embodiments, R₁ and R₂ each independently represent a lower alkyl or a halogen; and R₃ represents a lower alkyl, an aryl, a hydroxyl group, —(CH₂)ₘ—COOH, —(CH₂)ₘ—NH₂, —(CH₂)ₘ—N—C(═NH)NH₂, —(CH₂)ₘ—C(═O)NH₂, —SH, or —(CH₂)ₘ—S—CH₃. In certain embodiments, R₁ and R₂ each independently represent methyl, ethyl or propyl. In certain embodiments, R₁ and R₂ each represent methyl.

In certain embodiments, R₃ represents lower alkyl, phenyl, hydroxyphenyl, indole, imidazole, hydroxyl, —COOH, —CH₂—COOH, —CH₂—CH₂—N—C(═NH)NH₂, —CH₂—C(═O)NH₂, —CH₂—CH₂—C(═O)NH₂, —SH, or —CH₂—S—CH₃.

In certain particular embodiments, the polypeptide analogues the invention have one to fifteen additional amino acid residues attached to the carboxy terminal end of the base amino sequence. The base amino acid sequence refers to the amino acid sequence (e.g., GLP-1 (9-36)) prior to modification with the one to fifteen additional amino acid residues. One or more of the added amino acid residues can be non-naturally occurring amino acid residues. As used herein, non-naturally-occurring amino acids are amino acids other than the 20 amino acids coded for in human DNA. In certain embodiments, non-naturally occurring amino acids suitable for use in the present invention are those having aryl-containing side chains. In certain embodiments, the non-naturally occurring amino acid is biphenylalanine

In certain embodiments, the additional amino acids are all naturally occurring (e.g., alpha-amino acid residues). The amino acid residues attached to the carboxy terminus of the base sequence are selected from residues 31-39 of exendin-4. Exendin-4 is a peptide hormone isolated from the saliva of Heloderma suspectum (Gila monster) that has glucose lowering activity in mammals. Exendin-4 also has a much longer biological half-life than GLP-1 and has been shown to extend the in vivo half life of native GLP-1 analogues, e.g., GLP-1 (7-36), as disclosed in WO 2007/030519 (incorporated herein by reference).

In a particular embodiment, the amino acid residue is Pro. In certain embodiments, the amino acid residues are three or more consecutive amino acid residues selected from amino acid residues 31-39 of exendin-4. In another particular embodiment, the amino acid residues are Pro-Ser-Ser. In a further embodiment, the amino acid residues are Pro-Ser-Ser-Gly-Ala-Pro-Pro-Pro-Ser (SEQ ID NO: 3).

In certain embodiments, the carboxy terminus of the polypeptide analogues of the invention is a carboxamide.

In certain embodiments of the present invention, the polypeptide analogue has the following amino acid sequence:

(SEQ ID NO: 4)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-NH₂,

wherein Xaa is beta-dimethylaspartate or tert-leucine.

In certain embodiments of the present invention, the polypeptide analogue has the following amino acid sequence:

(SEQ ID NO: 5)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Asn-NH₂,

wherein Xaa is beta-dimethylaspartate or tert-leucine.

In certain embodiments of the present invention, the polypeptide analogue has the following amino acid sequence:

(SEQ ID NO: 6)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-NH₂,

wherein Xaa is beta-dimethylaspartate or tert-leucine.

In certain embodiments of the present invention, the polypeptide analogue has the following amino acid sequence:

(SEQ ID NO: 7)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Yaa-NH₂,

wherein Xaa is beta-dimethylaspartate or tert-leucine; and Yaa is biphenylalanine

In certain embodiments of the present invention, the polypeptide analogue has the following amino acid sequence:

(SEQ ID NO: 8)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-NH₂,

wherein Xaa is beta-dimethylaspartate or tert-leucine.

In certain embodiments of the present invention, the polypeptide analogue has the following amino acid sequence:

(SEQ ID NO: 9)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-Ser-Ser-NH₂,

wherein Xaa is beta-dimethylaspartate or tert-leucine.

In certain embodiments of the present invention, the polypeptide analogue has the following amino acid sequence:

(SEQ ID NO: 10)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-Ser-Ser-Gly-Ala-Pro-Pro-Pro-
Ser-NH₂,

wherein Xaa is beta-dimethylaspartate or tert-leucine.

In certain embodiments of the present invention, the polypeptide analogue has the following amino acid sequence:

(SEQ ID NO: 11)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-NH₂.

In certain embodiments of the present invention, the polypeptide analogue has the following amino acid sequence:

(SEQ ID NO: 12)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Asn-NH₂.

In certain embodiments of the present invention, the polypeptide analogue has the following amino acid sequence:

(SEQ ID NO: 13)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-NH₂.

In certain embodiments of the present invention, the polypeptide analogue has the following amino acid sequence:

(SEQ ID NO: 14)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Yaa-NH₂,

wherein Yaa is biphenylalanine

In certain embodiments of the present invention, the polypeptide analogue has the following amino acid sequence:

(SEQ ID NO: 15)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-NH₂.

In certain embodiments of the present invention, the polypeptide analogue has the following amino acid sequence:

(SEQ ID NO: 16)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-Ser-Ser-NH₂.

In certain embodiments of the present invention, the polypeptide analogue has the following amino acid sequence:

(SEQ ID NO: 17)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-Ser-Ser-Gly-Ala-Pro-Pro-Pro-
Ser-NH₂.

In certain embodiments, the polypeptide analogue is retro-inverso and comprises D-amino acids assembled in reversed order along the peptide chain.

In certain embodiments, the polypeptide analogue comprises D-allo amino acids.

In certain embodiments, the polypeptide analogue has only allo amino acids at positions corresponding to D-threonine and D-isoleucine.

Another aspect of the present invention provides formulations comprising any of the polypeptide analogues of the invention and pharmaceutically acceptable excipients. Exemplary formulations may comprise one or more of the polypeptide analogues described herein.

Another aspect of the invention relates to using the polypeptide analogues disclosed herein as part of a treatment regimen for various heart-related ailments or cardiac dysfunction. Exemplary heart-related ailments include myocardial infarction, ischemia-reperfusion injury, congestive heart failure, and cardiac arrest. The subject GLP-1 analogues can also be used in the prevention of heart related ailments. To more explicitly illustrate the applicability of the polypeptide analogues of the invention in methods of treating a variety of cardiac-related diseases and conditions, we provide the following non-limiting examples:

In certain embodiments, the present invention relates to a method for treating cardiac dysfunction by administering to a mammal in need thereof a therapeutically effective amount of a polypeptide analogue of the invention.

In certain embodiments, the present invention relates to a method for treating muscle dysfunction by administering to a mammal in need thereof a therapeutically effective amount of a polypeptide analogue according to the present invention.

In another embodiment, the present invention relates to a method for protecting the heart against ischemia-reperfusion injury by administering to a mammal in need thereof a therapeutically effective amount of a polypeptide analogue according to the present invention.

In another embodiment, the present invention relates to a method for treating congestive heart failure, comprising the step of administering to a mammal in need thereof a therapeutically effective amount of a polypeptide analogue of the invention.

Another aspect of the present invention relates to a method of enhancing myocardial glucose uptake by administering to a mammal in need thereof a therapeutically effective amount of a polypeptide analogue according to the present invention.

Yet another aspect of the present invention is a method of lowering fasting blood glucose in a mammal afflicted with diabetes by administering to mammal a therapeutically effective amount of a polypeptide analogue according to the present invention.

In certain embodiments, the present invention relates to the aforementioned methods, wherein the mammal is a primate, bovine, ovine, equine, porcine, rodent, feline or canine.

In certain embodiments, the present invention relates to the aforementioned methods, wherein the mammal is a human.

### DEFINITIONS

The term “amino acid” is intended to embrace all compounds, whether natural or synthetic, which include both an amino functionality and an acid functionality, including amino acid analogues and derivatives. In certain embodiments, the amino acids contemplated in the present invention are those naturally occurring amino acids found in proteins, or the naturally occurring anabolic or catabolic products of such amino acids, which contain amino and carboxyl groups. Naturally occurring amino acids are identified throughout by the conventional three-letter and/or one-letter abbreviations, corresponding to the trivial name of the amino acid, in accordance with the following list. The abbreviations are accepted in the peptide art and are recommended by the IUPAC-IUB commission in biochemical nomenclature.

By the term “amino acid residue” is meant an amino acid. In general the abbreviations used herein for designating the naturally occurring amino acids are based on recommendations of the IUPAC-IUB Commission on Biochemical Nomenclature (see Biochemistry (1972) 11:1726-1732). For instance Met, Ile, Leu, Ala and Gly represent “residues” of methionine, isoleucine, leucine, alanine and glycine, respectively. By the residue is meant a radical derived from the corresponding α-amino acid by eliminating the OH portion of the carboxyl group and the H portion of the α-amino group.

The term “amino acid side chain” is that part of an amino acid residue exclusive of the backbone, as defined by K. D. Kopple, “Peptides and Amino Acids”, W. A. Benjamin Inc., New York and Amsterdam, 1966, pages 2 and 33; examples of such side chains of the common amino acids are —CH₂CH₂SCH₃ (the side chain of methionine), —CH₂(CH₃)—CH₂CH₃ (the side chain of isoleucine), —CH₂CH(CH₃)₂ (the side chain of leucine) or H— (the side chain of glycine). These sidechains are pendant from the backbone Cα carbon.

“Heart-related ailments” or “cardiac dysfunction” includes any chronic or acute pathological event involving the heart and/or associated tissue (e.g., the pericardium, aorta and other associated blood vessels), including ischemia-reperfusion injury; congestive heart failure; cardiac arrest; myocardial infarction; cardiotoxicity caused by compounds such as drugs (e.g., doxorubicin, herceptin, thioridazine and cisapride); cardiac damage due to parasitic infection (bacteria, fimgi, rickettsiae, and viruses, e.g., syphilis, chronic Trypanosoma cruzi infection); fulminant cardiac amyloidosis; heart surgery; heart transplantation; traumatic cardiac injury (eg., penetrating or blunt cardiac injury, and aortic valve rapture), surgical repair of a thoracic aortic aneurysm; a suprarenal aortic aneurysm; cardiogenic shock due to myocardial infarction or cardiac failure; neurogenic shock and anaphylaxis.

The term “tetra-substituted Cβ carbon” refers to a carbon atom which is (i) directly pendant from the Cα carbon of the amino acid backbone, and (ii) includes four pendant substituents (including the Cα carbon), none of which is hydrogen.

The term “peptide,” as used herein, refers to a sequence of amino acid residues linked together by peptide bonds or by modified peptide bonds. The term “peptide” is intended to encompass peptide analogues, peptide derivatives, peptidomimetics and peptide variants. The term “peptide” is understood to include peptides of any length.

The term “polypeptide analogue” as used herein may refer not only to a peptide containing various natural amino acid substitutions to a base sequence but also to a peptide comprising one or more non-naturally occurring amino acid. Examples of non-naturally occurring amino acids include, but are not limited to, D-amino acids (i.e., an amino acid of an opposite chirality to the naturally occurring form), N-α-methyl amino acids, C-α-methyl amino acids, β-methyl amino acids, β-alanine (β-Ala), norvaline (Nva), norleucine (Nle), 4-aminobutyric acid (γ-Abu), 2-aminoisobutyric acid (Aib), 6-aminohexanoic acid (ε-Ahx), ornithine (orn), hydroxyproline (Hyp), sarcosine, citrulline, cysteic acid, cyclohexylalanine, α-amino isobutyric acid, t-butylglycine, t-butylalanine, 3-aminopropionic acid, 2,3-diaminopropionic acid (2,3-diaP), D- or L-phenylglycine, D- or L-2-naphthylalanine (2-Nal), 1,2,3,4-tetrahydroisoquinoline-3-carboxylic acid (Tic), D- or L-2-thienylalanine (Thi), D- or L-3-thienylalanine, D- or L-1-, 2-, 3- or 4-pyrenylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylalanine, D- or L-p-methoxybiphenylalanine, methionine sulphoxide (MSO) and homoarginine (Har). Other examples include D- or L-2-indole(alkyl)alanines and D- or L-alkylalanines, wherein alkyl is substituted or unsubstituted methyl, ethyl, propyl, hexyl, butyl, pentyl, isopropyl, iso-butyl, or iso-pentyl, and phosphono- or sulfated (e.g., —SO₃H) non-carboxylate amino acids.

Other examples of non-naturally occurring amino acids include 3-(2-chlorophenyl)-alanine, 3-chloro-phenylalanine, 4-chloro-phenylalanine, 2-fluoro-phenylalanine, 3-fluoro-phenylalanine, 4-fluoro-phenylalanine, 2-bromo-phenylalanine, 3-bromo-phenylalanine, 4-bromo-phenylalanine, homophenylalanine, 2-methyl-phenylalanine, 3-methyl-phenylalanine, 4-methyl-phenylalanine, 2,4-dimethyl-phenylalanine, 2-nitro-phenylalanine, 3-nitro-phenylalanine, 4-nitro-phenylalanine, 2,4-dinitro-phenylalanine, 1,2,3,4-Tetrahydroisoquinoline-3-carboxylic acid, 1,2,3,4-tetrahydronorharman-3-carboxylic acid, 1-naphthylalanine, 2-naphthylalanine, pentafluorophenylalanine, 2,4-dichloro-phenylalanine, 3,4-dichloro-phenylalanine, 3,4-difluoro-phenylalanine, 3,5-difluoro-phenylalanine, 2,4,5-trifluoro-phenylalanine, 2-trifluoromethyl-phenylalanine, 3-trifluoromethyl-phenylalanine, 4-trifluoromethyl-phenylalanine, 2-cyano-phenyalanine, 3-cyano-phenyalanine, 4-cyano-phenyalanine, 2-iodo-phenyalanine, 3-iodo-phenyalanine, 4-iodo-phenyalanine, 4-methoxyphenylalanine, 2-aminomethyl-phenylalanine, 3-aminomethyl-phenylalanine, 4-aminomethyl-phenylalanine, 2-carbamoyl-phenylalanine, 3-carbamoyl-phenylalanine, 4-carbamoyl-phenylalanine, m-tyrosine, 4-amino-phenylalanine, styrylalanine, 2-amino-5-phenyl-pentanoic acid, 9-anthrylalanine, 4-tert-butyl-phenylalanine, 3,3-diphenylalanine, 4,4′-diphenylalanine, benzoylphenylalanine, α-methyl-phenylalanine, α-methyl-4-fluoro-phenylalanine, 4-thiazolylalanine, 3-benzothienylalanine, 2-thienylalanine, 2-(5-bromothienyl)-alanine, 3-thienylalanine, 2-furylalanine, 2-pyridylalanine, 3-pyridylalanine, 4-pyridylalanine, 2,3-diaminopropionic acid, 2,4-diaminobutyric acid, allylglycine, 2-amino-4-bromo-4-pentenoic acid, propargylglycine, 4-aminocyclopent-2-enecarboxylic acid, 3-aminocyclopentanecarboxylic acid, 7-amino-heptanoic acid, dipropylglycine, pipecolic acid, azetidine-3-carboxylic acid, cyclopropylglycine, cyclopropylalanine, 2-methoxy-phenylglycine, 2-thienylglycine, 3-thienylglycine, α-benzyl-proline, α-(2-fluoro-benzyl)-proline, α-(3-fluoro-benzyl)-proline, α-(4-fluoro-benzyl)-proline, α-(2-chloro-benzyl)-proline, α-(3-chloro-benzyl)-proline, α-(4-chloro-benzyl)-proline, α-(2-bromo-benzyl)-proline, α-(3-bromo-benzyl)-proline, α-(4-bromo-benzyl)-proline, α-phenethyl-proline, α-(2-methyl-benzyl)-proline, α-(3-methyl-benzyl)-proline, α-(4-methyl-benzyl)-proline, α-(2-nitro-benzyl)-proline, α-(3-nitro-benzyl)-proline, α-(4-nitro-benzyl)-proline, α-(1-naphthalenylmethyl)-proline, α-(2-naphthalenylmethyl)-proline, α-(2,4-dichloro-benzyl)-proline, α-(3,4-dichloro-benzyl)-proline, α-(3,4-difluoro-benzyl)-proline, α-(2-trifluoromethyl-benzyl)-proline, α-(3-trifluoromethyl-benzyl)-proline, α-(4-trifluoromethyl-benzyl)-proline, α-(2-cyano-benzyl)-proline, α-(3-cyano-benzyl)-proline, α-(4-cyano-benzyl)-proline, α-(2-iodo-benzyl)-proline, a-(3-iodo-benzyl)-proline, α-(4-iodo-benzyl)-proline, α-(3-phenyl-allyl)-proline, α-(3-phenyl-propyl)-proline, α-(4-tert-butyl-benzyl)-proline, α-benzhydryl-proline, α-(4-biphenylmethyl)-proline, α-(4-thiazolylmethyl)-proline, α-(3-benzo[b]thiophenylmethyl)-proline, α-(2-thiophenylmethyl)-proline, α-(5-bromo-2-thiophenylmethyl)-proline, α-(3-thiophenylmethyl)-proline, α-(2-furanylmethyl)-proline, α-(2-pyridinylmethyl)-proline, α-(3-pyridinylmethyl)-proline, α-(4-pyridinylmethyl)-proline, α-allyl-proline, α-propynyl-proline, γ-benzyl-proline, γ-(2-fluoro-benzyl)-proline, γ-(3-fluoro-benzyl)-proline, γ-(4-fluoro-benzyl)-proline, γ-(2-chloro-benzyl)-proline, γ-(3-chloro-benzyl)-proline, γ-(4-chloro-benzyl)-proline, γ-(2-bromo-benzyl)-proline, γ-(3-bromo-benzyl)-proline, γ-(4-bromo-benzyl)-proline, γ-(2-methyl-benzyl)-proline, γ-(3-methyl-benzyl)-proline, γ-(4-methyl-benzyl)-proline, γ-(2-nitro-benzyl)-proline, γ-(3-nitro-benzyl)-proline, γ-(4-nitro-benzyl)-proline, γ-(1-naphthalenylmethyl)-proline, γ-(2-naphthalenylmethyl)-proline, γ-(2,4-dichloro-benzyl)-proline, γ-(3,4-dichloro-benzyl)-proline, γ-(3,4-difluoro-benzyl)-proline, γ-(2-trifluoromethyl-benzyl)-proline, γ-(3-trifluoromethyl-benzyl)-proline, γ-(4-trifluoromethyl-benzyl)-proline, γ-(2-cyano-benzyl)-proline, γ-(3-cyano-benzyl)-proline, γ-(4-cyano-benzyl)-proline, γ-(2-iodo-benzyl)-proline, γ-(3-iodo-benzyl)-proline, γ-(4-iodo-benzyl)-proline, γ-(3-phenyl-allyl-benzyl)-proline, γ-(3-phenyl-propyl-benzyl)-proline, γ-(4-tert-butyl-benzyl)-proline, γ-benzhydryl-proline, γ-(4-biphenylmethyl)-proline, γ-(4-thiazolylmethyl)-proline, γ-(3-benzothioienylmethyl)-proline, γ-(2-thienylmethyl)-proline, γ-(3-thienylmethyl)-proline, γ-(2-furanylmethyl)-proline, γ-(2-pyridinylmethyl)-proline, γ-(3-pyridinylmethyl)-proline, γ-(4-pyridinylmethyl)-proline, γ-allyl-proline, γ-propynyl-proline, trans-4-phenyl-pyrrolidine-3-carboxylic acid, trans-4-(2-fluoro-phenyl)-pyrrolidine-3-carboxylic acid, trans-4-(3-fluoro-phenyl)-pyrrolidine-3-carboxylic acid, trans-4-(4-fluoro-phenyl)-pyrrolidine-3-carboxylic acid, trans-4-(2-chloro-phenyl)-pyrrolidine-3-carboxylic acid, trans-4-(3-chloro-phenyl)-pyrrolidine-3-carboxylic acid, trans-4-(4-chloro-phenyl)-pyrrolidine-3-carboxylic acid, trans-4-(2-bromo-phenyl)-pyrrolidine-3-carboxylic acid, trans-4(3-bromo-phenyl)-pyrrolidine-3-carboxylic acid, trans-4(4-bromo-phenyl)-pyrrolidine-3-carboxylic acid, trans-4-(2-methyl-phenyl)-pyrrolidine-3-carboxylic acid, trans-4-(3-methyl-phenyl)-pyrrolidine-3-carboxylic acid, trans-4-(4-methyl-phenyl)-pyrrolidine-3-carboxylic acid, trans-4-(2-nitro-phenyl)-pyrrolidine-3-carboxylic acid, trans-4-(3-nitro-phenyl)-pyrrolidine-3-carboxylic acid, trans-4-(4-nitro-phenyl)-pyrrolidine-3-carboxylic acid, trans-4-(1-naphthyl)-pyrrolidine-3-carboxylic acid, trans-4-(2-naphthyl)-pyrrolidine-3-carboxylic acid, trans-4-(2,5-dichloro-phenyl)-pyrrolidine-3-carboxylic acid, trans-4-(2,3-dichloro-phenyl)-pyrrolidine-3-carboxylic acid, trans-4(2-trifluoromethyl-phenyl)-pyrrolidine-3-carboxylic acid, trans-4-(3-trifluoromethyl-phenyl)-pyrrolidine-3-carboxylic acid, trans-4-(4-trifluoromethyl-phenyl)-pyrrolidine-3-carboxylic acid, trans-4-(2-cyano-phenyl)-pyrrolidine-3-carboxylic acid, trans-4-(3-cyano-phenyl)-pyrrolidine-3-carboxylic acid, trans-4-(4-cyano-phenyl)-pyrrolidine-3-carboxylic acid, trans-4-(2-methoxy-phenyl)-pyrrolidine-3-carboxylic acid, trans-4-(3-methoxy-phenyl)-pyrrolidine-3-carboxylic acid, trans-4-(4-methoxy-phenyl)-pyrrolidine-3-carboxylic acid, trans-4-(2-hydroxy-phenyl)-pyrrolidine-3-carboxylic acid, trans-4-(3-hydroxy-phenyl)-pyrrolidine-3-carboxylic acid, trans-4-(4-hydroxy-phenyl)-pyrrolidine-3-carboxylic acid, trans-4(2,3-dimethoxy-phenyl)-pyrrolidine-3-carboxylic acid, trans-4(3,4-dimethoxy-phenyl)-pyrrolidine-3-carboxylic acid, trans-4-(3,5-dimethoxy-phenyl)-pyrrolidine-3-carboxylic acid, trans-4-(2-pyridinyl)-pyrrolidine-3-carboxylic acid, trans-4-(3-pyridinyl)-pyrrolidine-3-carboxylic acid, trans-4-(6-methoxy-3-pyridinyl)-pyrrolidine-3-carboxylic acid, trans-4(4-pyridinyl)-pyrrolidine-3-carboxylic acid, trans-4-(2-thienyl)-pyrrolidine-3-carboxylic acid, trans-4-(3-thienyl)-pyrrolidine-3-carboxylic acid, trans-4-(2-furanyl)-pyrrolidine-3-carboxylic acid, trans-4-isopropyl-pyrrolidine-3-carboxylic acid, 4-phosphonomethyl-phenylalanine, benzyl-phosphothreonine, (1′-amino-2-phenyl-ethyl)oxirane, (1′-amino-2-cyclohexyl-ethyl)oxirane, (1′-amino-2-[3-bromo-phenyl]ethyl)oxirane, (1′-amino-2-[4-(benzyloxy)phenyl]ethyl)oxirane, (1′-amino-2-[3,5-difluoro-phenyl]ethyl)oxirane, (1′-amino-2-[4-carbamoyl-phenyl]ethyl)oxirane, (1′-amino-2-[benzyloxy-ethyl])oxirane, (1′-amino-2-[4-nitro-phenyl]ethyl)oxirane, (1′-amino-3-phenyl-propyl)oxirane, (1′-amino-3-phenyl-propyl)oxirane, and/or salts and/or protecting group variants thereof.

As used herein, “protein” is a polymer consisting essentially of any of the 20 amino acids. Although “polypeptide” is often used in reference to relatively large proteins, and “peptide” is often used in reference to small protein, usage of these terms in the art overlaps and is varied. Unless evident from the context, the terms “peptide(s)”, “protein(s)” and “polypeptide(s)” are used interchangeably herein.

The terms “percent (%) amino acid sequence identity” or “percent amino acid sequence homology” or “percent (%) identical” as used herein with respect to a reference polypeptide is defined as the percentage of amino acid residues in a candidate peptide sequence that are identical with the amino acid residues in the reference polypeptide sequence after aligning the sequences and introducing gaps, if necessary, to achieve the maximum percent sequence identity, without considering any conservative substitutions as part of the sequence identity. Alignment for the purpose of determining percent amino acid sequence identity can be achieved by various techniques known in the art, for instance, using publicly available computer software such as ALIGN or Megalign (DNASTAR). Those skilled in the art can determine appropriate parameters for measuring alignment, including any algorithms needed to achieve maximal alignment over the full length of the peptide sequence being used in the comparison. For example, in the context of the present invention, an analogue of GLP-1 is said to share “substantial homology” with GLP-1 if the amino acid sequence of said compound is at least about 80%, at least about 90%, at least about 95%, or at least about 99% identical to native GLP-1.

The phrase “pharmaceutically acceptable” is employed herein to refer to those ligands, materials, compositions, and/or dosage forms which are, within the scope of sound medical judgment, suitable for use in contact with the tissues of human beings and animals, substantially non-pyrogenic, without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the inhibitor(s). These salts can be prepared in situ during the final isolation and purification of the inhibitor(s), or by separately reacting a purified inhibitor(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an inhibitor(s). These salts can likewise be prepared in situ during the final isolation and purification of the inhibitor(s), or by separately reacting the purified inhibitor(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

A “therapeutically effective amount” of a compound, e.g., such as a polypeptide or peptide analogue of the present invention, with respect to use in treatment, refers to an amount of the polypeptide or peptide in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “alkyl” refers to a fully saturated branched or unbranched carbon chain radical having the number of carbon atoms specified, or up to 30 carbon atoms if no specification is made. For example, a “lower alkyl” refers to an alkyl having from 1 to 10 carbon atoms, such as methyl, ethyl, propyl, butyl, pentyl, hexyl, heptyl, and octyl, and those which are positional isomers of these alkyls. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6, or 7 carbons in the ring structure.

Unless the number of carbons is otherwise specified, “lower alkyl”, as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “carbocycle”, as used herein, refers to an aromatic or non-aromatic ring in which each atom of the ring is carbon.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

“Alkenyl” refers to any branched or unbranched unsaturated carbon chain radical having the number of carbon atoms specified, or up to 26 carbon atoms if no limitation on the number of carbon atoms is specified; and having 1 or more double bonds in the radical. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl and tetracosenyl, in their various isomeric forms, where the unsaturated bond(s) can be located anywhere in the radical and can have either the (Z) or the (E) configuration about the double bond(s).

The term “alkynyl” refers to hydrocarbyl radicals of the scope of alkenyl, but having one or more triple bonds in the radical.

The terms “alkoxyl” or “alkoxy” as used herein refers to an alkyl group, as defined below, having an oxygen radical attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₁, where m and R₁ are described below.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, or the like.

The term “alkylthio” refers to an alkyl group, as defined above, having a sulfur radical attached thereto. In preferred embodiments, the “alkylthio” moiety is represented by one of —(S)-alkyl, —(S)-alkenyl, —(S)-alkynyl, and —(S)—(CH₂)ₘ—R₁, wherein m and R₁ are defined below. Representative alkylthio groups include methylthio, ethylthio, and the like.

As used herein, the term “nitro” means —NO₂; the term “halogen” designates F, Cl, Br or I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formulae:

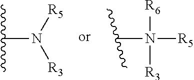

wherein R₃, R₅ and R₆ each independently represent a hydrogen, an alkyl, an alkenyl, —(CH₂)ₘ—R₁, or R₃ and R₅ taken together with the N atom to which they are attached complete a heterocycle having from 4 to 8 atoms in the ring structure; R₁ represents an alkenyl, aryl, cycloalkyl, a cycloalkenyl, a heterocyclyl or a polycyclyl; and m is zero or an integer in the range of 1 to 8. In preferred embodiments, only one of R₃ or R₅ can be a carbonyl, e.g., R₃, R₅ and the nitrogen together do not form an imide. In even more preferred embodiments, R₃ and R₅ (and optionally R₆) each independently represent a hydrogen, an alkyl, an alkenyl, or —(CH₂)ₘ—R₁. Thus, the term “alkylamine” as used herein means an amine group, as defined above, having a substituted or unsubstituted alkyl attached thereto, i.e., at least one of R₃ and R₅ is an alkyl group. In certain embodiments, an amino group or an alkylamine is basic, meaning it has a pKₐ>7.00. The protonated forms of these functional groups have pKₐs relative to water above 7.00.

The term “carbonyl” is art-recognized and includes such moieties as can be represented by the general formula:

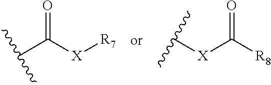

wherein X is a bond or represents an oxygen or a sulfur, and R₇ represents a hydrogen, an alkyl, an alkenyl, —(CH₂)ₘ—R₁ or a pharmaceutically acceptable salt, R₈ represents a hydrogen, an alkyl, an alkenyl or —(CH₂)ₘ—R₁, where m and R₁ are as defined above. Where X is an oxygen and R₇ or R₈ is not hydrogen, the formula represents an “ester”. Where X is an oxygen, and R₇ is as defined above, the moiety is referred to herein as a carboxyl group, and particularly when R₇ is a hydrogen, the formula represents a “carboxylic acid”. Where X is an oxygen, and R₈ is hydrogen, the formula represents a “formate”. In general, where the oxygen atom of the above formula is replaced by sulfur, the formula represents a “thiocarbonyl” group. Where X is a sulfur and R₇ or R₈ is not hydrogen, the formula represents a “thioester” group. Where X is a sulfur and R₇ is hydrogen, the formula represents a “thiocarboxylic acid” group. Where X is a sulfur and R₈ is hydrogen, the formula represents a “thioformate” group. On the other hand, where X is a bond, and R₇ is not hydrogen, the above formula represents a “ketone” group. Where X is a bond, and R₇ is hydrogen, the above formula represents an “aldehyde” group.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety that can be represented by the general formula:

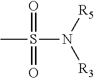

in which R₃ and R₅ are as defined above.

The term “sulfate” is art recognized and includes a moiety that can be represented by the general formula:

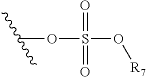

in which R₇ is as defined above.

The term “sulfamido” is art recognized and includes a moiety that can be represented by the general formula:

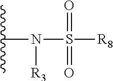

in which R₂ and R₄ are as defined above.

The term “sulfonate” is art-recognized and includes a moiety that can be represented by the general formula:

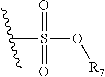

in which R₇ is an electron pair, hydrogen, alkyl, cycloalkyl, or aryl.

The terms “sulfoxido” or “sulfinyl”, as used herein, refers to a moiety that can be represented by the general formula:

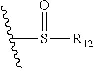

in which R₁₂ is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

A “patient” or “subject” to be treated by the subject method can mean either a human or non-human subject.

The term “interact” as used herein is meant to include all interactions (e.g., biochemical, chemical, or biophysical interactions) between molecules, such as protein-protein, protein-nucleic acid, nucleic acid-nucleic acid, protein-small molecule, nucleic acid-small molecule, or small molecule-small molecule interactions.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “retro modified,” as used herein, refers to a peptide that is made up of L-amino acids in which the amino acid residues are assembled in the opposite direction to the native peptide with respect to which it is retro modified (see FIG. 1).

The term “inverso modified,” as used herein, refers to a peptide that is made up of D-amino acids in which the amino acid residues are assembled in the same direction as the native peptide with respect to which it is inverso modified (see FIG. 1).

The term “retro-inverso modified,” as used herein, refers to a peptide that is made up of D-amino acids in which the amino acid residues are assembled in the opposite direction to the native peptide with respect to which it is retro-inverso modified (see FIG. 1).

Polypeptide analogues can differ from the native peptides by amino acid sequence or by modifications that do not affect the sequence or both. Certain analogues include peptides whose sequences differ from the wild-type sequence (i.e., the sequence of the homologous portion of the naturally occurring peptide) only by conservative amino acid substitutions, preferably by only one, two, or three, substitutions; for example, differing by substitution of one amino acid for another with similar characteristics (e.g., valine for glycine, arginine for lysine) or by one or more non-conservative amino acid substitutions, deletions, or insertions, which do not abolish the peptide's biological activity. Modifications that do not usually alter primary sequence include in vivo or in vitro chemical derivatization of peptides (e.g., acetylation or carboxylation). Also included are modifications of glycosylation, e.g., those made by modifying the glycosylation patterns of a peptide during its synthesis and processing or in further processing steps, e.g., by exposing the peptide to enzymes (e.g., mammalian glycosylating or deglycosylating enzymes) that affect glycosylation. Also included are sequences that have phosphorylated amino acid residues, e.g., phosphotyrosine, phosphoserine, or phosphotreonine. The invention also includes analogues in which one or more peptide bonds have been replaced with an alternative type of covalent bond (a “peptide mimetic”), which is less susceptible to cleavage by peptidases. Where proteolytic degradation of the peptides following injection into a subject is a problem, replacement of a particularly sensitive peptide bond with a non-cleavable peptide mimetic will make the resulting peptide more stable and thus likely to be more useful as a therapeutic agent. Such amino acid mimetics, and methods of incorporating them into peptides, are well known in the art. Protecting groups are also useful.

Native peptide sequences set out herein are written according to the generally accepted convention whereby the N-terminal amino acid is on the left, and the C-terminal amino acid is on the right. The sequences of the peptide analogues, however, may run in the same direction as that of the corresponding sequence in the native peptide (i.e., the N-terminus of the peptide analogue corresponds to the N-terminal end of the corresponding amino acid sequence in the native peptide), or the sequence of the peptide may be inverted (i.e., the N-terminus of the peptide analogue corresponds to the C-terminal end of the corresponding amino acid sequence in the native peptide). For example, for a peptide region having a sequence from N- to C-terminus: 123456, the sequence of a retro-modified peptide corresponding to this region would be from N- to C-terminus: 654321, or could be optionally represented from C-terminus to N-terminus as 123456, so long as the termini are clearly identified in the depiction (see, e.g., FIG. 1).

As noted above, certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis or by derivation with a chiral auxiliary, where the resulting diastereomeric mixture is separated and the auxiliary group cleaved to provide the pure desired enantiomer. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomer.

For purposes of this invention, the chemical elements are identified in accordance with the Periodic Table of the Elements, CAS version, Handbook of Chemistry and Physics, 67th ed., 1986-87, inside cover.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### C-Terminal Modifications to GLP-1 (9-36)

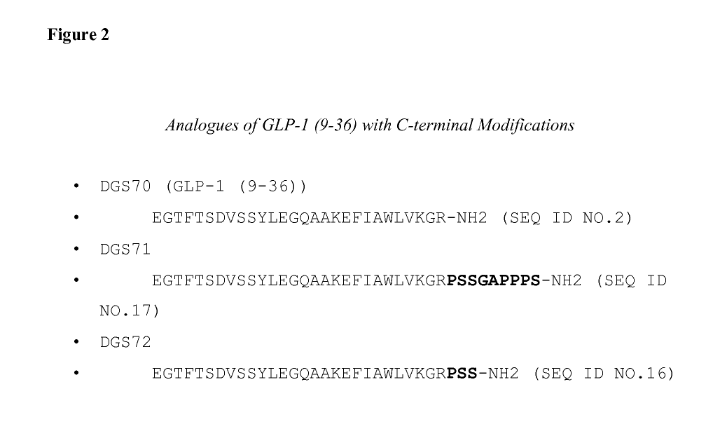

GLP-1 (9-36) analogues containing C-terminal extensions (FIG. 2) were tested in 9 week old male Sprague Dawley rats to evaluate biological half-life as well as other pharmacokinetic properties of the analogues. Rats used in the study were acclimated to laboratory conditions for approximately 1 week prior to testing.

Polypeptide analogues were prepared as solutions for intravenous administration (bolus injection in the jugular vein) at concentrations of 0.15 mg/mL and 1.5 mg/mL in appropriate buffers. The compounds were then injected while the animals were still under anesthesia. Blood samples were taken form each animal at no more than 5 occasions. Following dose administration, blood samples (0.3 to 0.4 mL) were obtained by jugular venipuncture (lithium heparin was used as an anticoagulant) at selected time points (0, 2.5, 5, 10 and 15 minutes or 30, 45, 60, 120 and 240 minutes). The samples were analyzed for parent drug by LC-MS/MS.

Numerical data was subjected to calculation of group mean values, standard deviations and coefficients of variation (expressed as a percent), where appropriate. The pharmacokinetic analysis (non-compartmental) of plasma concentration was performed using the PhAST software program (Version 2.3-004, Pheonix International Life Sciences Inc.). The highest experimental concentration was considered the peak concentration (Cₘₐₓ; observed value). Whenever possible, the observed terminal phase rate constant (Kₑₗ) was calculated from the terminal 3 or more points (non-zero and non-Cmax points) of the log-linear regression. The number of points included in the regression analysis was such as to optimize r² value calculated for the regression. Terminal phase half-life (t_{1/2}) was determined by dividing 0.693 by Kₑₗ. The area under the plasma concentration of each compound versus time-curve from time zero to the last quantifiable concentration (AUC₀₋ₜ) was calculated by the linear trapezoidal method (Bailer, A. J., (1988) J. Pharmacokin. Biopharm., 1, 303-309). AUC_{0-∞}, the area under the plasma concentration versus time curve from time zero to infinity, was calculated as the sum of AUC₀₋ₜ plus the ratio of the last plasma concentration to Kₑₗ. Values below the limit of quantification were assigned a value of zero for pharmacokinetic analysis. The resulting data are presented in Table 1 below.

TABLE 1
Pharmacokinetic Parameters in Plasma Following A Single Bolus Intravenous
Administration of Different Compounds to Male Sprague Dawley Rats

| Compound | Group | Dose Level | Pharmacokinetic Parameters | | | | | | | |
|---|---|---|---|---|---|---|---|---|---|---|
| Name | No. | (mg/kg) | Kₑₗ | AUC_{0-∞} | AUC₀₋ₜ | Cₘₐₓ | Tₘₐₓ | T_{1/2} | CL | Vdss |
| DGS70 | 9^{a} | 0.15 | 0.422 | 3908.62 | 3825.80 | 1063.843 | 2.50 | 1.64 | 38.38 | 136.88 |
| | 10 | 1.5 | 0.313 | 40712.70 | 40315.18 | 10609.650 | 2.50 | 2.22 | 36.84 | 144.73 |
| DGS71 | 11 | 0.15 | 0.051 | 19215.92 | 18430.17 | 1921.890 | 2.50 | 13.63 | 7.81 | 124.33 |
| | 12 | 1.5 | 0.038 | 169622.31 | 168672.97 | 15276.280 | 2.50 | 18.26 | 8.84 | 151.93 |
| DGS72 | 13 | 0.15 | 0.139 | 10389.70 | 10279.91 | 2100.803 | 2.50 | 4.99 | 14.44 | 88.24 |
| | 14 | 1.5 | 0.071 | 110352.05 | 110182.41 | 19285.180 | 2.50 | 9.72 | 13.59 | 84.65 |

^{a}Results should be interpreted with caution as only 3 data points available.
AUC_{0-∞}: The area under the plasma concentration versus time curve from time zero to infinity (ng · min/mL)
AUC₀₋ₜ: The area under the plasma concentration versus time-curve from time zero to last time point (ng · min/mL)
Cₘₐₓ: The highest observable concentration (ng/mL)
Kel: Elimination rate constant (min⁻¹)
Tₘₐₓ: Time to Cmax (min)
t?: Terminal phase half-life (min)
CL: Clearance (mL/min kg)
Vdss: Volume of distribution at steady state (mL/kg)
Untruncated values were used for calculation purposes

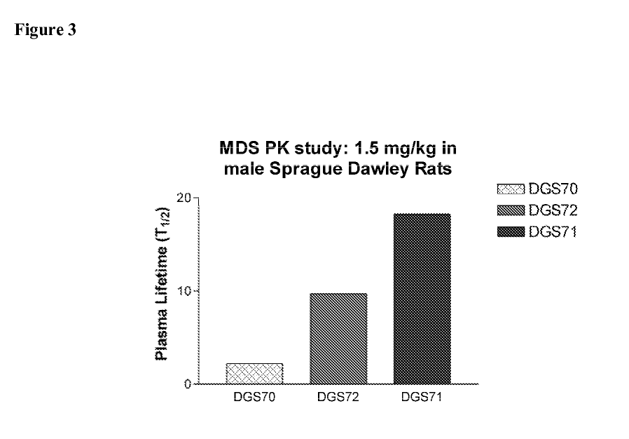

The half-life of unmodified GLP-1 (9-36) (DGS70) is between 1.6 and 2.2 minutes, whereas polypeptide analogues containing C-terminal extensions from residues 31-39 of exendin-4 (DGS71 and DGS72) have much longer half-lives. DGS72, which contains a 3 amino acid C-terminal extension, has a half-life of between 4.99 and 9.72. The GLP-1 polypeptide analogue DGS71, with an additional 9 amino acids from exendin-4 appended to the C-terminus, has a half-life of between 13.63 and 18.26. (FIG. 3) Thus, longer lived polypeptide analogues of GLP-1 can be produced through extending the C-terminus of the base peptide.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

All of the U.S. patents and U.S. patent application publications cited herein are hereby incorporated by reference.

1. A polypeptide comprising:
a base amino acid sequence at least 90% identical to GLP-1 (9-34) or GLP-1 (9-36) (SEQ ID NOS: 1 and 2), wherein the analogue has a longer in vivo half-life than GLP-1 (9-34) or GLP-1 (9-36).

2. A polypeptide analogue comprising:
a) a base amino acid sequence at least 90% identical to GLP-1 (9-34) or GLP-1 (9-36) (SEQ ID NOS: 1 and 2); and
b) one to fifteen amino acid residues attached to the carboxy terminus of the base amino acid sequence,
wherein the analogue has a longer in vivo half-life than GLP-1 (9-34) or GLP-1 (9-36).

3-6. (canceled)

7. A polypeptide analogue comprising:
a base amino acid sequence at least 90% identical to GLP-1 (9-34) or GLP-1 (9-36) (SEQ ID NOS: 1 and 2); wherein the amino acid residue corresponding to position 9 of GLP-1 is an amino acid analogue having a tetrasubstituted C_{β} carbon; and the analogue has longer in vivo half-life than GLP-1 (9-34) or GLP-1 (9-36).

8. A polypeptide analogue comprising:
a) a base amino acid sequence at least 90% identical to one of GLP-1 (9-34), GLP-1 (9-36), (SEQ ID NOS: 1 and 2); wherein the amino acid residue corresponding to position 9 of GLP-1 is an amino acid analogue having a tetrasubstituted C_{β} carbon; and
b) one to fifteen amino acid residues attached to the carboxy terminus of the base amino acid sequence,
wherein the analogue has a longer in vivo half-life than GLP-1 (9-34) or GLP-1 (9-36).

9. The polypeptide analogue of claim 7 or 8, wherein the amino acid residue corresponding to position 9 of GLP-1 is represented by the following formula: 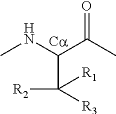
wherein:
R₁ and R₂ each independently represent a lower alkyl, heteroalkyl, cycloalkyl, heterocycloalkyl, aryl, alkoxyl, carbonyl, carboxamide, halogen, hydroxyl, amine, or cyano, or R₁ and R₂ taken together form a ring of 4-7 atoms;
R₃ represents a lower alkyl, a heteroalkyl, amino, alkoxyl, halogen, carboxamide, carbonyl, cyano, thiol, thioalkyl, acylamino, nitro, azido, sulfate, sulfonate, sulfonamido, —(CH₂)ₘ—R₄, —(CH₂)ₘ—OH, —(CH₂)ₘ—COOH, —(CH₂)ₘ—O-lower alkyl, —(CH₂)ₘ—O-lower alkenyl, —(CH₂)ₙ—O—(CH₂)ₘ—R₄, —(CH₂)ₘ—S-lower alkyl, —(CH₂)ₘ—S-lower alkenyl, —(CH₂)ₙ—S—(CH₂)ₘ—R₄, —(CH₂)ₘ—N—C(═NH)NH₂, —(CH₂)ₘ—C(═O)NH₂, or —(CH₂)ₘ—NH₂;
R₄ represents, independently for each occurrence, an aryl, aralkyl, cycloalkyl, cycloalkenyl, or non-aromatic heterocyclyl; and
m is 0, 1 or 2.

10. The polypeptide analogue of claim 9, wherein R₁ and R₂ each independently represent a lower alkyl or a halogen; and R₃ represents a lower alkyl, an aryl, a hydroxyl group, —(CH₂)ₘ—COOH, —(CH₂)ₘ—NH₂, —(CH₂)ₘ—N—C(═NH)NH₂, —(CH₂)ₘC(═O)NH₂, —SH, or —(CH₂)ₘ—S—CH₃.

11-13. (canceled)

14. The polypeptide analogue of claim 2 or 8, wherein a non-naturally occurring amino acid residue is attached to the carboxy terminus of the base amino acid sequence.

15. The polypeptide analogue of claim 14 wherein the non-naturally occurring amino acid residue has an aryl-containing side chain.

16. The polypeptide analogue of claim 14, wherein the non-naturally occurring amino acid is biphenylalanine.

17. The polypeptide analogue of claim 2 or 8, wherein the amino acid residues attached to the carboxy terminus of the base amino acid sequence are selected from amino acid residues 31-39 of exendin-4.

18-22. (canceled)

23. The polypeptide analogue of claim 7, wherein said analogue has the following amino acid sequence:
(SEQ ID NO: 4)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-NH₂,
wherein Xaa is beta-dimethylaspartate or tert-leucine.

24. The polypeptide analogue of claim 7, wherein said analogue has the following amino acid sequence:
(SEQ ID NO: 5)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Asn-NH₂,
wherein Xaa is beta-dimethylaspartate or tert-leucine.

25. The polypeptide analogue of claim 7, wherein said analogue has the following amino acid sequence:
(SEQ ID NO: 6)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-NH₂,
wherein Xaa is beta-dimethylaspartate or tert-leucine.

26. The polypeptide analogue of claim 8, wherein said analogue has the following amino acid sequence:
(SEQ ID NO: 7)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Yaa-NH₂,
wherein Xaa is beta-dimethylaspartate or tert-leucine; and Yaa is biphenylalanine.

27. The polypeptide analogue of claim 8, wherein said analogue has the following amino acid sequence:
(SEQ ID NO: 8)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-NH₂,
wherein Xaa is beta-dimethylaspartate or tert-leucine.

28. The polypeptide analogue of claim 8, wherein said analogue has the following amino acid sequence:
(SEQ ID NO: 9)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-Ser-Ser-NH₂,
wherein Xaa is beta-dimethylaspartate or tert-leucine.

29. The polypeptide analogue of claim 8, wherein said analogue has the following amino acid sequence:
(SEQ ID NO: 10)
Xaa-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-Ser-Ser-Gly-Ala-Pro-Pro-Pro-
Ser-NH₂,
wherein Xaa is beta-dimethylaspartate or tert-leucine.

30. The polypeptide analogue of claim 1, wherein said analogue has the following amino acid sequence:
(SEQ ID NO: 11)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-NH₂.

31. The polypeptide analogue of claim 1, wherein said analogue has the following amino acid sequence:
(SEQ ID NO: 12)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Asn-NH₂.

32. The polypeptide analogue of claim 1, wherein said analogue has the following amino acid sequence:
(SEQ ID NO: 13)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-NH₂.

33. The polypeptide analogue of claim 2, wherein said analogue has the following amino acid sequence:
(SEQ ID NO: 14)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Yaa-NH₂,
wherein Yaa is biphenylalanine.

34. The polypeptide analogue of claim 2, wherein said analogue has the following amino acid sequence:
(SEQ ID NO: 15)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-NH₂.

35. The polypeptide analogue of claim 2, wherein said analogue has the following amino acid sequence:
(SEQ ID NO: 16)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-Ser-Ser-NH₂.

36. The polypeptide analogue of claim 2, wherein said analogue has the following amino acid sequence:
(SEQ ID NO: 17)
Glu-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Lys-Gly-Arg-Pro-Ser-Ser-Gly-Ala-Pro-Pro-Pro-
Ser-NH₂.

37-40. (canceled)

41. A method for treating cardiac dysfunction, comprising the step of administering to a mammal in need thereof a therapeutically effective amount of a polypeptide analogue according to claim 1.

42-48. (canceled)


SEQUENCE LISTING (17 sequences)

SEQ ID NO: 1 - protein, 26 aa, Unknown
  Description of Unknown Glucagon-like peptide 1
EGTFTSDVSSYLEGQAAKEFIAWLVK

SEQ ID NO: 2 - protein, 28 aa, Unknown
  Description of Unknown Glucagon-like peptide 1
EGTFTSDVSSYLEGQAAKEFIAWLVKGR

SEQ ID NO: 3 - protein, 9 aa, Heloderma suspectum
PSSGAPPPS

SEQ ID NO: 4 - protein, 26 aa, Artificial Sequence
  Description of Artificial Sequence Synthetic peptide
XGTFTSDVSSYLEGQAAKEFIAWLVK

SEQ ID NO: 5 - protein, 27 aa, Artificial Sequence
  Description of Artificial Sequence Synthetic peptide
XGTFTSDVSSYLEGQAAKEFIAWLVKN

SEQ ID NO: 6 - protein, 28 aa, Artificial Sequence
  Description of Artificial Sequence Synthetic peptide
XGTFTSDVSSYLEGQAAKEFIAWLVKGR

SEQ ID NO: 7 - protein, 29 aa, Artificial Sequence
  Description of Artificial Sequence Synthetic peptide
XGTFTSDVSSYLEGQAAKEFIAWLVKGRX

SEQ ID NO: 8 - protein, 29 aa, Artificial Sequence
  Description of Artificial Sequence Synthetic peptide
XGTFTSDVSSYLEGQAAKEFIAWLVKGRP

SEQ ID NO: 9 - protein, 31 aa, Artificial Sequence
  Description of Artificial Sequence Synthetic polypeptide
XGTFTSDVSSYLEGQAAKEFIAWLVKGRPSS

SEQ ID NO: 10 - protein, 37 aa, Artificial Sequence
  Description of Artificial Sequence Synthetic polypeptide
XGTFTSDVSSYLEGQAAKEFIAWLVKGRPSSGAPPPS

SEQ ID NO: 11 - protein, 26 aa, Artificial Sequence
  Description of Artificial Sequence Synthetic peptide
EGTFTSDVSSYLEGQAAKEFIAWLVK

SEQ ID NO: 12 - protein, 27 aa, Artificial Sequence
  Description of Artificial Sequence Synthetic peptide
EGTFTSDVSSYLEGQAAKEFIAWLVKN

SEQ ID NO: 13 - protein, 28 aa, Artificial Sequence
  Description of Artificial Sequence Synthetic peptide
EGTFTSDVSSYLEGQAAKEFIAWLVKGR

SEQ ID NO: 14 - protein, 29 aa, Artificial Sequence
  Description of Artificial Sequence Synthetic peptide
EGTFTSDVSSYLEGQAAKEFIAWLVKGRX

SEQ ID NO: 15 - protein, 29 aa, Artificial Sequence
  Description of Artificial Sequence Synthetic peptide
EGTFTSDVSSYLEGQAAKEFIAWLVKGRP

SEQ ID NO: 16 - protein, 31 aa, Artificial Sequence
  Description of Artificial Sequence Synthetic polypeptide
EGTFTSDVSSYLEGQAAKEFIAWLVKGRPSS

SEQ ID NO: 17 - protein, 37 aa, Artificial Sequence
  Description of Artificial Sequence Synthetic polypeptide
EGTFTSDVSSYLEGQAAKEFIAWLVKGRPSSGAPPPS